SEQUENCE LISTING (4 sequences)

SEQ ID NO: 1 - protein, 393 aa, Artificial sequence
  This is a fusion protein sequence containing Interferon-beta and a portion of human immunoglobulin heavy chain sequence
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1179 bp, Artificial sequence
  DNA encoding SEQ ID NO 1
atgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacgacaaaactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccatcctccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - protein, 422 aa, Artificial sequence
  This is a fusion protein sequence containing interferon-beta and a portion of human immunoglubulin heavy chain sequence
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRNEFAGAAAVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1266 bp, Artificial sequence
  DNA encoding SEQ ID NO 3
atgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatattgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaacgagttcgccggcgccgctgcggtcgacaaaactcacacatgcccaccgtgcccagctccggaactcctgggcggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

(19) United States
(12) Patent Application Publication
(10) Pub. No.: US 2010/0249381 A1
(43) Pub. Date: Sep. 30, 2010

(54) Method for Purifying FC-Fusion Proteins

(76) Inventors: David Delvaille, Pessac (FR); Thierry Ziegler, Leognan (FR); Alex Eon-Duval, Vevey (CH)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/738,447

(22) PCT Filed: Oct. 21, 2008
(86) PCT No.: PCT/EP08/64208

§ 371 Date: Apr. 16, 2010

Related U.S. Application Data
(60) Provisional application No. 61/007,134, filed on Dec. 11, 2007.

(30) Foreign Application Priority Data
Oct. 22, 2007 (EP) 07118982.3

(51) Int. Cl. C07K 16/00; C07K 1/18

(52) U.S. Cl. 530/387.3; 530/416

(57) ABSTRACT

The invention relates to a method for the purification of Fc-fusion via blue dye affinity chromatography, in particular for the reduction of the amount of free Fc-moieties in an Fc-fusion proteins preparation.

### FIELD OF THE INVENTION

The present invention is in the field of protein purification. More specifically, it relates to the purification of an Fc-fusion proteins preparation containing a single protein fused to an Fc region, in particular for the reduction of the amount of free Fc-moieties in said preparation via blue dye affinity chromatography. The single protein fused to an Fc region is preferably a fusion protein able to bind to a blue dye affinity resin, and more preferably a fusion protein comprising IFN-β fused to an Fc region.

### BACKGROUND OF THE INVENTION

Proteins have become commercially important as drugs that are generally called “biologicals”. One of the greatest challenges is the development of cost effective and efficient processes for purification of proteins on a commercial scale. While many methods are now available for large-scale preparation of proteins, crude products, such as cell culture supernatants, contain not only the desired product but also impurities, which are difficult to separate from the desired product. Although cell culture supernatants of cells expressing recombinant protein products may contain fewer impurities if the cells are grown in serum-free medium, the host cell proteins (HCPs) still remain to be eliminated during the purification process. Additionally, the health authorities request high standards of purity for proteins intended for human administration.

A number of chromatographic methods are known that are widely used for protein purification. Methods such as affinity chromatography and the like have been widely used.

Affinity chromatography is based on the affinity of a protein of interest to another protein that is immobilized to a chromatography resin. Examples for such immobilized ligands are the bacterial cell wall proteins Protein A and Protein G, having specificity to the Fc portion of certain immunoglobulins (Igs). Although both Protein A and Protein G have a strong affinity for IgG antibodies, they have varying affinities to other immunoglobulin classes and isotypes as well.

Blue dye affinity chromatography is based on a dye-ligand, Cibacron Blue, which is bound to a matrix (e.g. sepharose or agarose). In the Blue Sepharose resin, the ligand Cibacron Blue F3G-A, is covalently coupled to sepharose™ through chlorotriazine ring (Vlatakis G et al., 1987). Blue Sepharose has been used for the purification of interferon beta (Knight E Jr and Fahey, 1981) and albumin. Examples of commercially available blue dye affinity matrices include Blue Sepharose 6FF resin (GE Healthcare), Blue Sepharose CL-6B (GE Healthcare), Blue Trisacryl M (Pall/BioSepra), Affi-Gel Blue (Bio-Rad) and Toyopearl AF-Blue (Tosoh Bioscience) or Cibacron Blue F3GA (Polysciences Inc.).

Ion exchange chromatography (IEC) systems are used for separation of proteins primarily on the basis of differences in charge.

Anion exchangers can be classified as either weak or strong. The charge group on a weak anion exchanger is a weak base, which becomes de-protonated and, therefore, loses its charge at high pH. DEAE-sepharose is an example of a weak anion exchanger, where the amino group can be positively charged below pH˜9 and gradually loses its charge at higher pH values. Diethylaminoethyl (DEAE) or diethyl-(2-hydroxy-propyl)aminoethyl (QAE) have chloride as counter ion, for instance. A strong anion exchanger, on the other hand, contains a strong base, which remains positively charged throughout the pH range normally used for ion exchange chromatography (pH 1-14). Q-sepharose (Q stands for quaternary ammonium) is an example for a strong anion exchanger.

Cation exchangers can also be classified as either weak or strong. A strong cation exchanger contains a strong acid (such as a sulphopropyl group) that remains charged from pH 1-14, whereas a weak cation exchanger contains a weak acid (such as a carboxymethyl group), which gradually loses its charge as the pH decreases below 4 or 5. Carboxymethyl (CM) and sulphopropyl (SP) have sodium as counter ion, for example.

Hydrophobic interaction chromatography (HIC) is used to separate proteins on the basis of hydrophobic interactions between the hydrophobic moieties of the protein and insoluble, immobilized hydrophobic groups on the matrix. Generally, the protein preparation in a high salt buffer is loaded on the HIC column. The salt in the buffer interacts with water molecules to reduce the salvation of the proteins in solution, thereby exposing hydrophobic regions in the protein which are then adsorbed by hydrophobic groups on the matrix. The more hydrophobic the molecule, the less salt is needed to promote binding. Usually, a decreasing salt gradient is used to elute proteins from a column. As the ionic strength decreases, the exposure of the hydrophilic regions of the protein increases and proteins elute from the column in order of increasing hydrophobicity.

Fc-fusion proteins are chimeric proteins comprising a protein or a biologically active domain thereof fused to the Fc region of an immunoglobulin that is frequently an immunoglobulin G (IgG). The Fc region, also called Fc fragment, of an immunoglobulin consists of two identical arms which comprise the hinge region (H) and the second (CH2) and third (CH3) domain of an antibody heavy chain. Fc-fusion proteins are widely used as therapeutics as they offer advantages conferred by the Fc region, such as:
the possibility of purification using protein A or protein G affinity chromatography with affinities which vary according to the IgG isotype. Human IgG1, IgG2 and IgG4 bind strongly to Protein A and all human IgGs including IgG3 bind strongly to Protein G;
an increased half-life in the circulatory system, since the Fc region binds to the salvage receptor FcRn which protects from lysosomal degradation;
depending on the medical use of the Fc-fusion protein, the Fc effector functions may be desirable. Such effector functions include antibody-dependent cellular cytotoxicity (ADCC) through interactions with Fc receptors (FcγRs) and complement-dependent cytotoxicity (CDC) by binding to the complement component 1q (C1q). IgG isoforms exert different levels of effector functions. Generally, human IgG1 and IgG3 have strong ADCC and CDC effects while human IgG2 exerts weak ADCC and CDC effects. Human IgG4 tends to display weak ADCC and no CDC effects.

Serum half-life and effector functions can be modulated by engineering the Fc region to increase or reduce its binding to FcRn, FcγRs and C1q respectively, depending on the therapeutic use intended for the Fc-fusion protein.

In ADCC, the Fc region of an antibody binds to Fc receptors (FcγRs) on the surface of immune effector cells such as natural killers and macrophages, leading to the phagocytosis or lysis of the targeted cells.

In CDC, the antibodies kill the targeted cells by triggering the complement cascade at the cell surface. IgG isoforms exert different levels of effector functions increasing in the order of IgG4<IgG2<IgG1≦IgG3. Human IgG1 displays high ADCC and CDC, and is the most suitable for therapeutic use against pathogens and cancer cells.

Under certain circumstances, for example when depletion of the target cell is undesirable, abrogating effector functions is required. On the contrary, in the case of Fc-fusion proteins intended for oncology use, increasing effector functions may improve their therapeutic activity (Carter et al., 2006).

Modifying effector functions can be achieved by engineering the Fc region to either improve or reduce binding of FcγRs or the complement factors.

The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Two regions of the CH2 domain are critical for FcγRs and complement C1q binding, and have unique sequences in IgG2 and IgG4. For instance, substitution of IgG2 residues at positions 233-236, according to EU index position as defined by Kabat et al. (Kabat, E. A., Wu, T. T., Perry, H. M., Gottesman, K. S., and Foeller, C. (1991), into human IgG1 greatly reduced ADCC and CDC (Armour et al., 1999 and Shields et al., 2001).

Numerous mutations have been made in the CH2 domain of IgG and their effect on ADCC and CDC was tested in vitro (Shields et al., 2001, Idusogie et al., 2001 and 2000, Steurer et al., 1995). In particular, the mutation E333A was reported to increase both ADCC and CDC (Idusogie et al., 2001 and 2000).

Increasing the serum half-life of a therapeutic Fc-fusion protein is another way to improve its efficacy, allowing higher circulating levels, less frequent administration and reduced doses. This can be achieved by enhancing the binding of the Fc region to neonatal FcR (FcRn). FcRn, which is expressed on the surface of endothelial cells, binds the IgG in a pH-dependent manner and protects it from degradation. Several mutations located at the interface between the CH2 and CH3 domains have been shown to increase the half-life of IgG1 (Hinton et al., 2004 and Vaccaro et al., 2005).

The following Table 1 summarizes some known mutations of the IgG Fc region (taken from Invivogen's website):

| Engineered | IgG | | | | |
|---|---|---|---|---|---|
| Fc | Isotype | Mutations | Properties | Potential Benefits | Applications |
| hIgG1e1 | human | T250Q/M428L | Increased | Improved | Vaccination; |
| | IgG1 | | plasma half- | localization to target; | therapeutic use |
| | | | life | increased efficacy; | |
| | | | | reduced dose or | |
| | | | | frequency of | |
| | | | | administration | |
| hIgG1e2 | human | M252Y/S254T/T256E + | Increased | Improved | Vaccination; |
| | IgG1 | H433K/N434F | plasma half- | localization to target; | therapeutic us |
| | | | life | increased efficacy; | |
| | | | | reduced dose or | |
| | | | | frequency of | |
| | | | | administration | |
| hIgG1e3 | human | E233P/L234V/L235A/ | Reduced | Reduced adverse | Therapeutic use |
| | IgG1 | ΔG236 + | ADCC and | events | without cell |
| | | A327G/A330S/P331S | CDC | | depletion |
| hIgG1e4 | human | E333A | Increased | Increased efficacy | Therapeutic use |
| | IgG1 | | ADCC and | | with cell |
| | | | CDC | | depletion |
| hIgG2e1 | human | K322A | Reduced CDC | Reduced adverse | Vaccination; |
| | IgG2 | | | events | therapeutic use |

In one class of Fc-fusion proteins having therapeutic utility, Fc regions have been fused to extracellular domains of certain receptors belonging to the tumor necrosis factor receptor (TNF-R) superfamily (Locksley et al., 2001, Bodmer et al., 2002, Bossen et al., 2006). A hallmark of the members of the TNFR family is the presence of cysteine-rich pseudo-repeats in the extracellular domain, as described e.g. by Naismith and Sprang, 1998.

Another example of Fc-fusion protein consists of an Fc region linked to a single interferon beta protein. Interferon beta (interferon-β, IFN-beta or IFN-β) is a naturally occurring soluble glycoprotein belonging to the class of cytokines. Interferons (IFNs) have a wide range of biological activities, such as anti-viral, anti-proliferative and immunomodulatory properties. Interferon beta is used as a therapeutic protein drug, a so-called biological, in a number of diseases, such as e.g. multiple sclerosis, cancer, or viral diseases such as e.g. SARS or hepatitis C virus infections.

Fusion proteins containing IFN-β as a biologically active molecule linked to an IgG Fc region are described in WO2005/001025.

Given the therapeutic utility of Fc-fusion proteins there is a need for significant amounts of purified protein that is adequate for human administration.

### SUMMARY OF THE INVENTION

The outcome of the production of proteins composed of two different subunits linked together (e.g. a single protein fused to an arm of an Fc region), is the formation of at least three different species, i.e. two homodimers (e.g. Fc arm/Fc arm and single protein-Fc arm/single protein-Fc arm) and one heterodimer (e.g. Fc arm/single protein-Fc arm).

Therefore, one of the problems that may be encountered during the production of a single protein fused to an Fc region (i.e. a single protein fused to an arm of an Fc region) is the generation of large amounts of “free Fc-moieties”, i.e. dimers of Fc arms (e.g. Fc arm/Fc arm homodimers) resulting from the expression of the Fc region not linked to any protein of interest or from proteolytic cleavage of the Fc-fusion proteins containing the protein of interest and in particular the single protein fused to the Fc region.

Object of the invention is the elimination of said free Fc-moieties that represent the major contaminant in the production of a single protein fused to an Fc region.

The present invention is based on the development of a purification method of a single protein fused to an Fc region from a fluid, composition or preparation of Fc-fusion proteins, by which the amount of free Fc-moieties that is present is reduced.

The invention, therefore, relates to a method for reducing the concentration of free Fc-moieties in a preparation of Fc-fusion proteins containing a single protein fused to an Fc region, the method comprising subjecting said preparation to blue dye affinity chromatography and eliminating the free Fc-moieties by washing the resin at a pH ranging from about 8.4 to about 8.9.

In a second aspect, the invention relates to the use of blue dye affinity chromatography for the reduction of free Fc-moieties in an Fc-fusion proteins preparation containing a single protein fused to an Fc region.

In a third aspect, the invention relates to purified Fc-fusion proteins containing a single protein fused to an Fc region and comprising less than about 5% or less than about 2% or less than about 1% of free Fc-moieties of the total protein concentration.

In a fourth aspect, the present invention relates to a method for the separation of a single protein fused to an Fc region from an Fc-fusion proteins preparation and from free Fc-moieties. In this aspect the single protein fused to an Fc region is able to bind to a blue dye affinity resin and the blue dye affinity chromatography is preceded and/or followed by one or more steps of affinity chromatography, anion exchange chromatography, cation exchange chromatography, hydrophobic interaction chromatography size exclusion chromatography, nanofiltration or ultrafiltration. Preferably, the single protein fused to an Fc region is a single INF-β fused to an Fc region.

In a fifth aspect, the invention relates to the use of blue dye affinity chromatography and one or more steps of affinity chromatography, cation exchange chromatography, anion exchange chromatography, hydrophobic interaction chromatography, size exclusion chromatography, nanofiltration, or ultrafiltration for the separation of a single protein fused to an Fc region from an Fc-fusion proteins preparation and from free Fc-moieties.

A sixth aspect of the invention relates to a single protein fused to an Fc region purified by the method according to the invention, preferably a single IFN-β fused to an Fc region.

A seventh aspect of the invention relates to a single IFN-β fused to an Fc region purified by the method according to the invention, in which the Fc region is a mutated Fc region.

### BRIEF DESCRIPTION OF THE DRAWINGS

FIG. 1 shows the chromatographic profile of the blue sepharose chromatography described in Step 2 of Example 1 (i) OD at 280 nm (mAU), (ii) Conductivity, (iii) pH. 1-Equilibration, 2-Load. 3-Wash 1, 4-Wash 2, 5-Elution.

FIG. 2 shows the RP-HPLC analysis spectrum of the load (FIG. 2.A), wash 2 (FIG. 2.B.), and eluate (FIG. 2.C) fractions from Step 2 of Example 1 where peak (a) represents free Fc, peak (b) Fcmut/IFNβ-Fcmut and peak (c) IFNβ-Fcmut/IFNβ-Fcmut.

FIG. 3 shows the chromatographic profile of the cation exchange chromatography described in Step 4 of Example 1 (i) OD at 280 nm (mAU), (ii) Conductivity, (iii) pH. 1-Equilibration, 2-Load. 3-Wash, 4-Elution, 5-Regeneration, 6-Sanitisation.

FIG. 4 shows the chromatographic profile of the hydrophobic interaction chromatography described in Step 5 of Example 1 (i) OD at 280 nm (mAU), (ii) Conductivity, (iii) pH. 1-Equilibration, 2-Load. 3-Wash 1, 4-Regeneration, 5-Sanitisation.

### BRIEF DESCRIPTION OF THE SEQUENCE LISTING

SEQ ID NO: 1 is an IFNβ-Fcmut amino acid sequence. Amino acids 1 to 166 represent the mature human interferon beta and amino acids 167 to 393 represent a portion of a mutated human immunoglobulin gamma heavy chain sequence.

SEQ ID NO: 2 is a polynucleotide coding for a polypeptide of SEQ ID NO: 1.

SEQ ID NO: 3 is an IFNβ-Fc arm amino acid sequence. Amino acids 1 to 21 represent IFN-β signal peptide, residues 22 to 187 mature IFN-β, residues 188 to 195 a linker sequence and residues 196 to 422 a portion of a human immunoglobulin heavy chain.

SEQ ID NO: 4 is a polynucleotide coding for a polypeptide of SEQ ID NO: 3.

### DETAILED DESCRIPTION OF THE INVENTION

The outcome of the production of proteins composed of two different subunits linked together, is the formation of at least three different species, i.e. two homodimers and one heterodimer. For instance, when producing INFβ-Fc fusion proteins, at least the following species are formed: Fc arm/Fc arm dimer (homodimer), INFβ-Fc arm/INFβ-Fc arm (homodimer) and Fc arm/IFNβ-Fc arm (heterodimer).

When a composition of Fc-fusion proteins (e.g. IFNβ-Fc arm/IFNβ-Fc arm and Fc arm/IFNβ-Fc arm) is desired, the concentration of the free Fc-moieties (e.g. Fc arm/Fc arm dimer), which could amount to 30% of the total, must be reduced.

The present invention is based on the finding that blue dye affinity chromatography can provide a convenient and simple way to efficiently reduce the amount or extent of free Fc-moieties that may be present in a fluid or composition of Fc-fusion proteins binding to a blue dye affinity resin, increasing thereby the purity of the Fc-fusion proteins.

The first aspect of the invention, therefore, relates to a method for purifying an Fc-fusion proteins preparation, containing a single protein fused to an Fc region, from free Fc-moieties present in a fluid comprising said Fc-fusion proteins, the method comprising the steps of:
(a) loading said fluid on a blue dye affinity chromatography resin;
(b) washing the resin with a buffer having a pH of about 8.4 to about 8.9 thereby eliminating the free Fc-moieties from the resin; and
(c) eluting the Fc-fusion proteins from the resin.

The Fc fusion proteins are able to bind to a blue dye affinity resin and the blue dye affinity chromatography resin.

The fluid comprising the Fc-fusion proteins may be any composition or preparation, such as e.g. a body fluid derived from a human or animal, or a fluid derived from a cell culture, such as e.g. a cell culture supernatant or cell culture harvest. It may also be a fluid derived from another purification step, such as e.g. the eluate or flow-through from a capture step or any other suitable purification step preceding the blue sepharose chromatography such as the eluate of protein A chromatography.

The fluid may preferably be cell culture material, e.g. solubilised cells, more preferably cell culture supernatant. The term “cell culture supernatant”, as used herein, refers to a medium in which cells are cultured and into which proteins are secreted provided they contain appropriate cellular signals, so-called signal peptides. It is preferred that the Fc-fusion proteins expressing cells are cultured under serum-free culture conditions. Thus, preferably, the cell culture supernatant is devoid of animal derived components. Most preferably, the cell culture medium is a chemically defined medium.

Herein, an Fc region may be referred to as an Fc fragment or Fc domain. Herein, the terms “Fc region”, “Fc fragment” or “Fc domain” are interchangeable and should be construed as having the same meaning.

The term “single protein fused to an Fc region” (e.g. Fc arm/IFNβ-Fc arm), as used herein, is meant to encompass fusion proteins comprising a non-immunoglobulin protein (e.g. IFN-β), which will be called herein the “therapeutic moiety” (irrespective of whether or not treatment of disease is intended) linked to only one arm of an Fc region-derived moiety, which will be called herein the “Fc-moiety”.

The term “Fc-fusion proteins”, as used herein, includes the “single protein fused to an Fc region” and the fusion proteins in which the therapeutic moiety consists of two copies of a non-immunoglobulin protein each of them linked to an arm of the Fc moiety (e.g. IFNβ-Fc arm/IFNβ-Fc arm). The Fc-fusion proteins to be purified in accordance with the present invention bind to a blue dye chromatography resin.

The term “Fc-moiety”, as used herein, refers to a dimer of at least one immunoglobulin constant domain, preferably human constant region, selected from the hinge, CH2, CH3, CH4 domain, or any combination thereof, and preferably a hinge, CH2 and CH3 domain. The immunoglobulin constant domain may be derived from any of IgG, IgA, IgE, IgM, IgD or combination or isotype thereof. Preferably, it is IgG, such as e.g. IgG1, IgG2, IgG3 or IgG4. More preferably, it is IgG1.

In accordance with the present invention, the Fc-moiety of the Fc-fusion proteins may also be modified in order to modulate effector functions.

For instance, the following Fc mutations, according to EU index positions (Kabat et al., 1991), can be introduced if the Fc-moiety is derived from IgG1:

T250Q/M428L

M252Y/S254T/T256E+H433K/N434F

E233P/L234V/L235A/ΔG236+A327G/A330S/P331S

E333A; K322A.

Therapeutic Fc-fusion proteins, i.e. Fc-fusion proteins intended for treatment or prevention of disease of an animal or preferably for human treatment or administration, are especially suitable to be purified in accordance with the invention.

Any Fc-fusion protein binding to a blue dye affinity resin may be purified in accordance with the present invention.

The therapeutic moiety of an Fc-fusion protein may e.g. be or be derived from EPO, TPO, Growth Hormone, Interferon-alpha, Interferon-beta, Interferon-gamma, PDGF-beta, VEGF, IL-1alpha, IL-1beta, IL-2, IL-4, IL-5, IL-8, IL-10, IL-12, IL-17, IL-18, IL-18 binding protein, TGF-beta, TNF-alpha, or TNF-beta.

The therapeutic moiety of an Fc-fusion protein may also be derived from a receptor, e.g. a transmembrane receptor, preferably be or be derived from the extracellular domain of a receptor, and in particular a ligand binding fragment of the extracellular part or domain of a given receptor. Examples for therapeutically interesting receptors are CD2, CD3, CD4, CD8, CD11a, CD11b, CD14, CD18, CD20, CD22, CD23, CD25, CD33, CD40, CD44, CD52, CD80, CD86, CD147, CD164, IL-2 receptor, IL-4 receptor, IL-6 receptor, IL-12 receptor, IL-17 receptors (IL-17R, IL-17RB (IL-17RH1), IL-17RC (IL-17RL), IL-17RD (hSEF) or IL-17RE), IL-18 receptor subunits (IL-18R-alpha, IL-18R-beta), EGF receptor, VEGF receptor, integrin alpha 4 10 beta 7, the integrin VLA4, B2 integrins, TRAIL receptors 1, 2, 3, and 4, RANK, RANK ligand, epithelial cell adhesion molecule (EpCAM), intercellular adhesion molecule-3 (ICAM-3), CTLA4 (which is a cytotoxic T lymphocyte-associated antigen), Fc-gamma-I, II or III receptor, HLA-DR 10 beta, HLA-DR antigen, L-selectin.

In line with the definition above, the Fc-fusion proteins of the invention may contain Fc-fusion heterodimers and homodimers. The “homodimer” comprises two copies of a single non-immunoglobulin protein each of them linked to an arm of the Fc-moiety (e.g. a dimer of two IgG disulfide-bridged hinge-CH2-CH3 arms). The “heterodimer” or “single protein fused to an Fc region” comprises an Fc moiety of which only one arm is fused to a single non-immunoglobulin protein.

Preferably, said heterodimer comprises a single IFN-β linked to one of the two IgG hinge-CH2-CH3 arms (see e.g. WO 2005/001025 and SEQ ID NO: 3). An heterodimer that contains two subunits, the first comprising a mutated IgG hinge-CH2-CH3 arm linked to a single IFN-β protein (i.e. IFNβ-Fc mut, corresponding to the amino acid sequence of SEQ ID: NO 1) and the second subunit comprising a mutated IgG hinge-CH2-CH3 arm (i.e. Fc mut, corresponding to the amino acids residues 167 to 393 of SEQ ID: NO 1), is also preferred in accordance with the present invention. Therefore, according to the present invention, a single IFN-β fused to an Fc moiety preferably comprising a polypeptide selected from:
(a) SEQ ID NO: 1 or 3;
(b) a polypeptide encoded by a polynucleotide hybridising to the complement of SEQ ID NO: 2 or 4 under highly stringent conditions; and
(c) a mutein of (a) having at least 80% or 85% or 90% or 95% sequence identity to the polypeptide of (a);
is suitable for purification in accordance with the method of the invention.

Such single IFN-β fused to an Fc moiety will be generally referred to a Fc arm/IFNβ-Fc arm in the frame of the present invention.

A further preferred Fc arm/IFNβ-Fc arm, in accordance with the present invention, comprises the mutation N297A according to EU index position corresponding to mutation N272A of SEQ ID NO: 3.

In accordance with the present invention, the Fc-fusion proteins are subjected to dye affinity chromatography in order to reduce, decrease, or eliminate free Fc-moieties, preferably at least to less than 5%, 2% or 1% of the total protein concentration of the Fc-fusion proteins composition.

The term “free Fc-moieties”, “free Fc moiety”, or simply “free Fc”, as used herein, is meant to encompass any part of the Fc-fusion proteins to be purified in accordance with the present invention, which corresponds to or is derived from the Fc-moiety without comprising complete further domains or complete sequences derived from the therapeutic moiety. Free Fc may e.g. contain dimers of the IgG hinge, CH2 and CH3 domains, which are not linked to significant portions of the therapeutic moiety.

Monomers derived from the Fc-moiety may also be contained in the free Fc fraction. It is understood that free Fc may still contain a number of amino acid residues from the therapeutic moiety, such as e.g. one to fifty or one to twenty, or one to ten, or one to five amino acids, or one single amino acid, belonging to the therapeutic moiety, still fused to the Fc-moiety.

In accordance with the present invention, the blue dye affinity chromatography may be carried out on any suitable resin. Preferably the resin comprises Cibacron Blue F3G-A ligand. Preferably, the blue dye affinity chromatography is carried out on Blue Sepharose resin. A resin commercially available under the name Blue Sepharose 6FF resin (GE Healthcare) is an example of an affinity resin that is particularly suitable for step (a) of the present method. The technical features of Blue Sepharose FF are as follows:

TECHNICAL SPECIFICATIONS

| | |
|---|---|
| Ligand | Cibacron Blue F3G-A |
| Ligand coupling method | Triazine coupling |
| Binding capacity | >18 mg human serum albumin/ml medium |
| Ligand density | ≈7 μmol Cibacron Blue/ml medium |
| Matrix | Highly cross-linked agarose, 6% |
| Average particle size | 90 μm |
| pH stability | 4-12 (long term), 3-13 (short term) |
| Chemical stability | 40° C. for 7 days in: 70% ethanol, 6 M |
| | guanidine hydrochloride, 8 M urea |

Other suitable commercially available blue dye affinity columns are selected from Blue Sepharose CL-6B (GE Healthcare), Blue Trisacryl M (Pall/BioSepra), Affi-Gel Blue (Bio-Rad), Econo-Pac blue cartridges (Bio-Rad), SwellGel Blue (Pierce), and Toyopearl AF-Blue (Tosoh Bioscience) or Cibacron Blue F3G-A (Polysciences Inc.).

In step (a) of the purification method of the invention, before loading the fluid comprising the Fc-fusion proteins on the blue dye resin, the fluid is preferably adjusted to a pH of less than 6 preferably about 4.5 or if necessary diluted with water to a conductivity of less than about 20 mS/cm at about pH 5. This allows binding of the Fc-fusion proteins to the blue dye resin.

In step (b) of the method of the invention, the free Fc-moieties are washed from the blue dye resin with a buffer having a pH of about 8.4 to about 8.9. The pH may e.g. be at about 8.4, 8.5, 8.6, 8.7, 8.8 or at about 8.9.

In step (b), free Fc-moieties are washed out from the blue dye affinity resin using any suitable buffer (e.g. ammonium acetate, Tris-HCl). An ammonium acetate buffer at a pH of 8.7±0.2 or 8.7±0.1 is preferred.

In a further preferred embodiment, the free Fc-moieties are washed from the blue sepharose column with an isocratic salt concentration ranging from 40 to 100 mM at about pH 8.7. The isocratic salt concentration can e.g. be 40, 45, 50, 55, 60, 65, 70, 75, 80, 85, 90, 95 or 100 mM ammonium acetate at pH of about 8.6 to about 8.8. It is preferably 50 mM ammonium acetate at pH 8.7±0.1. A Tris-HCl buffer may also be used preferably at a concentration of 50 mM at pH 8.5.

In step (c) of the method of the invention, the Fc-fusion proteins are eluted from the blue dye affinity chromatography column.

The elution from the dye affinity chromatography column of the Fc-fusion proteins in step (c) is carried out in a buffer with a pH ranging from about 8.3 to 8.7. In a preferred embodiment, the elution is carried out in a buffer comprising ammonium acetate, NaCl, propylene glycol to which ammonia is added to adjust the pH. Propylene glycol is preferably used in a concentration ranging between 10%-50%, 10-40%, 20%-40%, 20%-30% and 40%-50%. Suitable buffer concentrations are e.g. selected from about 25 mM, or about 50 mM or about 100 mM or about 150 mM or about 200 mM or about 250 mM.

In a highly preferred embodiment, the method of the invention is used as a second step of a purification scheme of Fc-fusion proteins wherein the fluid loaded in step (a) on the blue sepharose resin is the eluate of Protein A or Protein G affinity chromatography to which a fluid comprising said Fc-fusion proteins was subjected first. The fluid may preferably be cell culture material, e.g. solubilized cells, more preferably cell culture supernatant.

The Protein A or G affinity chromatography is preferably used as a capture step, and thus serves for the purification of the Fc-fusion proteins, in particular for the elimination of host cell proteins (HCPs) and Fc-fusion protein aggregates, and for the concentration of the Fc-fusion proteins preparation.

A column commercially available under the name MabSelect Xtra (GE Healthcare) is an example of an affinity resin that is particularly suitable for the capture step.

In accordance with the present invention, the eluate of the blue sepharose chromatography of step (c) may then be used for further purification e.g. as described in further detail below.

In a preferred embodiment of the invention, step (a) comprises loading the blue sepharose resin at a maximum dynamic capacity of about 40 g of Fc-fusion proteins per litre of packed blue dye affinity resin.

In addition, the blue sepharose chromatography of the invention reduces the levels of free Fc-moieties to less than about 5% or less than about 2% or less than about 1% of free Fc-moieties. Therefore, the blue sepharose chromatography step of the invention reduces the levels of free Fc-moieties to below detection levels as determined by SDS-PAGE. Therefore, in a preferred embodiment of the invention, the eluate of the blue dye chromatography has levels of free Fc-moieties that are undetectable by SDS-PAGE under non-reducing conditions and silver staining when loading 1 μg of Fc-fusion proteins.

A second aspect of the invention relates to the use of blue dye affinity chromatography for the reduction of the concentration of free Fc-moieties in a composition comprising Fc-fusion proteins, in particular Fc-fusion proteins able to bind to a blue dye affinity resin and in particular Fc-fusion proteins containing a single protein fused to an Fc region e.g. Fc arm/IFNβ-Fc arm. Preferably, the blue dye resin is blue sepharose resin.

A third aspect of the invention relates to purified Fc-fusion proteins containing a single protein fused to an Fc region and comprising less than about 5% or less than about 2% or less than about 1% of free Fc-moieties of the total protein concentration.

A fourth aspect of the invention relates to a method for the separation of a single protein fused to an Fc region (e.g. Fc arm/IFNβ-Fc arm) from an Fc-fusion proteins preparation (e.g. IFNβ-Fc arm/IFNβ-Fc arm and Fc arm/IFNβ-Fc arm) and from free Fc-moieties (e.g. Fc arm/Fc arm dimers). In this process, the blue dye affinity sepharose chromatography may be used in a purification method having one or more additional steps, preferably selected from affinity chromatography, anion exchange chromatography, cation exchange chromatography, hydrophobic interaction chromatography and size exclusion chromatography. Filtration steps such as nanofiltration or ultrafiltration can be used between one or more of the purification steps.

In one embodiment, the fluid (i.e. Fc-fusion proteins preparation) containing the single protein fused to an Fc region, e.g. Fc arm/IFNβ-Fc arm, to be purified is subjected to an initial capture step e.g. on Protein A. The eluate of the capture step may then be applied onto a blue dye affinity chromatography according to the invention.

The eluate of the blue dye affinity chromatography, comprising Fc-fusion proteins and a reduced concentration of free Fc-moieties, may then be subjected to further chromatography steps for example cation exchange chromatography followed by hydrophobic interaction chromatography (e.g. in Example 1).

Preferably, the resin used in the cation exchange chromatography is a strong cation exchange resin. A column commercially available under the name Fractogel SO₃— (Merck) is an example of a cation exchange resin that is particularly suitable in the context of the present method.

Preferably, the eluate from the blue dye affinity chromatography step is diluted or dialysed into an appropriate loading buffer before loading it on the cation exchange column. Prior to loading, the cation exchange column is preferably equilibrated with an appropriate equilibration buffer. An appropriate equilibration/washing buffer is e.g. a sodium acetate, NaCl buffer at pH 5.0±0.5 and with a conductivity of 7.5±0.5 mS/cm. It is preferably a sodium acetate, NaCl buffer at pH 5.0±0.2 and a conductivity of 7.5±0.2 mS/cm.

The cation exchange column is washed with the equilibration/washing buffer and the Fc-fusion proteins are then eluted from the cation exchange resin with e.g. a buffer at 35 mM to 45 mM sodium phosphate and 50 to 150 mM KCl buffer at pH 7.0±0.5 and a conductivity of 16±1 mS/cm. It is preferably a 40 mM sodium phosphate and 100 mM KCl buffer at pH 7.0±0.1 with a conductivity of 16±0.5 mS/cm.

The eluate from the cation exchange chromatography step, containing mainly the single protein fused to an Fc region (e.g. Fc arm/IFNβ-Fc arm), is then subjected to hydrophobic interaction chromatography. The hydrophobic interaction chromatography may be carried out on any suitable resin. A resin commercially available under the name PPG-600M (Tosoh) is a resin particularly suitable for the hydrophobic interaction chromatography step according to the present method.

The hydrophobic interaction chromatography column is preferably equilibrated with an appropriate equilibration buffer.

The eluate from the cation exchange chromatography can be diluted or dialysed into an appropriate loading buffer before loading it on the hydrophobic exchange column. Ammonium sulphate is preferably added to the eluate of the cation exchange column before loading.

After loading, the column is washed with an appropriate wash buffer, and the purified single protein fused to an Fc region (e.g. Fc arm/IFNβ-Fc arm) is collected in the flow through. The flow through is preferably collected when a major rise of a peak of absorbance at 280 nm appears and until a stable baseline is reached at the end of the wash.

An appropriate equilibration/wash buffer is e.g. a buffer at 35 mM to 45 mM sodium phosphate, 0.1 to 0.8 M ammonium sulphate, at pH: 6.8±0.8 with a conductivity of 5.3±8 mS/cm. It is preferably a 40 mM sodium phosphate, 0.5M ammonium sulphate buffer at pH 6.8±0.2 with a conductivity of 5.3±2 mS/cm.

The flow through comprising the purified single protein fused to an Fc region (e.g. Fc arm/IFNβ-Fc arm) can be subjected to further steps of nanofiltration and dialysis.

In another embodiment, the fluid (i.e. Fc-fusion proteins preparation) containing the single protein fused to an Fc region (e.g. Fc arm/IFNβ-Fc arm) to be purified is subjected to an initial capture step e.g. on Protein A. The eluate of the capture step is then applied onto a blue dye affinity chromatography according to the invention. The eluate of the blue dye affinity chromatography, comprising Fc-fusion proteins and a reduced concentration of free Fc-moieties, may also be subjected to further chromatography steps for example anion exchange chromatography followed by a size exclusion chromatography.

The anion exchange chromatography may be carried out on any suitable anion exchange resin, such as e.g. weak or strong anion exchangers as explained above in the Background of the Invention. Preferably, the anion exchange chromatography is carried out on a strong anion exchange resin. A resin commercially available under the name Q Sepharose Fast Flow (GE Healthcare) is an example of an anion exchange resin that is particularly suitable for the anion exchange chromatography according to the present method.

Preferably, the eluate from the blue dye affinity chromatography step is diluted or dialysed into an appropriate loading buffer before loading it on the anion exchange column. The anion exchange column is also preferably equilibrated with the loading buffer. The same buffer is preferably used to wash the column.

An appropriate equilibration/loading/washing buffer is e.g. a buffer containing 25 to 100 mM Tris at a pH 8.5±0.4. It is preferably Tris at 50 mM and at pH 8.5.

The eluate from the anion exchange column, containing mainly the single protein fused to an Fc region (e.g. Fc arm/IFNβ-Fc arm), is obtained by applying a salt gradient reaching up to 0.5 M NaCl+Tris 50 mM pH 8.5.

The size exclusion chromatography may be carried out on any suitable gel. A gel commercially available under the name Superdex 200 (GE Healthcare) is an example of a size exclusion gel that is particularly suitable. The eluate from the anion exchange chromatography can be further diluted or dialysed into an appropriate loading buffer before loading it on the size exclusion column. Ammonium sulphate is preferably added to the eluate of the cation exchange column before loading on the size exclusion column.

After loading, the column is washed with an appropriate wash buffer, and the purified single protein fused to an Fc region (e.g. Fc arm/IFNβ-Fc arm) is collected in the flow through. The flow through is preferably collected when a major rise of a peak of absorbance at 280 nm appears and until a stable baseline is reached at the end of the wash.

The volume of the resin, the length and diameter of the column to be used, as well as the dynamic capacity and flow-rate depend on several parameters such as the volume of fluid to be treated, concentration of protein in the fluid to be subjected to the process of the invention, etc. Determination of these parameters for each step is well within the average skills of the person skilled in the art.

In a preferred embodiment of the present purification process, one or more ultrafiltration or nanofiltration steps are performed. Ultrafiltration or nanofiltration are useful for removal of small organic molecules and salts in the eluate resulting from previous chromatographic steps, to equilibrate the Fc-fusion proteins in the bulk buffer, or to concentrate the Fc-fusion proteins to the desired concentration. Ultrafiltration may e.g. be performed on ultrafiltration membranes, with pore sizes allowing the removal of components having molecular weights below 5, 10, 15, 20, 25, 30 or more kDa.

If the protein purified according to the process of the invention is intended for administration to humans, it is advantageous to include one or more steps of virus removal in the process.

In order to facilitate storage or transport, for instance, the material may be frozen and thawed before and/or after any purification step of the invention.

In accordance with the present invention, the Fc-fusion proteins may be produced in eukaryotic expression systems, such as yeast, insect, or mammalian cells, resulting in glycosylated Fc-fusion proteins.

In accordance with the present invention, it is most preferred to express the Fc-fusion proteins in mammalian cells such as animal cell lines, or in human cell lines. Chinese hamster ovary (CHO) cells or the murine myeloma cell line NS0 are examples of cell lines that are particularly suitable for expression of the Fc-fusion proteins to be purified. The Fc-fusion proteins can also preferably be produced in human cell lines, such as e.g. the human fibrosarcoma HT1080 cell line, the human retinoblastoma cell line PERC6, or the human embryonic kidney cell line 293, or a permanent amniocyte cell line as described e.g. in EP1230354.

If the Fc-fusion proteins to be purified are expressed by mammalian cells secreting it, the starting material of the purification process of the invention is cell culture supernatant, also called harvest or crude harvest. If the cells are cultured in a medium containing animal serum, the cell culture supernatant also contains serum proteins as impurities.

Preferably, the Fc-fusion proteins expressing and secreting cells are cultured under serum-free conditions. The Fc-fusion proteins may also be produced in a chemically defined medium. In this case, the starting material of the purification process of the invention is serum-free cell culture supernatant that mainly contains host cell proteins as impurities. If growth factors are added to the cell culture medium, such as insulin, for example, these proteins will be eliminated during the purification process as well.

In order to create soluble, secreted Fc-fusion proteins, that are released into the cell culture supernatant, either the natural signal peptide of the therapeutic moiety of the Fc-fusion proteins is used, or a heterologous signal peptide, i.e. a signal peptide derived from another secreted protein being efficient in the particular expression system used, such as e.g. the bovine or human Growth Hormone signal peptide, or the immunoglobulin signal peptide.

The term “muteins”, as used herein, refers to analogs (e.g. Fcmut/INFβ-Fc mut) of Fc arm/INFβ-Fc arm preferably having a subunit (i.e. INFβ-Fcmut) corresponding to the sequence of SEQ ID NO: 1, in which one or more of the amino acid residues of IFNβ-Fc arm are replaced by different amino acid residues, or are deleted, or one or more amino acid residues are added to the original sequence of IFN-β or Fc arm without changing considerably the activity of the resulting products as compared with the original IFNβ-Fc arm/Fc arm. These muteins are prepared by known synthesis and/or by site-directed mutagenesis techniques, or any other known technique suitable therefore.

Muteins in accordance with the present invention include proteins encoded by a nucleic acid, such as DNA or RNA, which hybridises to DNA or RNA, which encodes a IFNβ-Fcmut according to SEQ ID NO: 1 under stringent conditions. An example for a DNA sequence encoding a IFNβ-Fcmut is SEQ ID NO: 2.

The term “stringent conditions” refers to hybridisation and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992). Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990).

In a preferred embodiment, any such mutein has at least 80%, at least 85%, at least 90%, or at least 95% identity or homology thereto.

Preferred changes for muteins in accordance with the present invention are what are known as “conservative” substitutions. Conservative amino acid substitutions of IFNβ-Fc arm may include synonymous amino acids within a group which have sufficiently similar physicochemical properties whereby substitution between members of the group continue to preserve the biological function of the molecule (Grantham, 1974). It is clear that insertions and deletions of amino acids may also be made in the above-defined sequences without altering their function, particularly if the insertions or deletions only involve a few amino acids, e.g., under thirty, under twenty, or preferably under ten, and do not remove or displace amino acids which are critical to a functional conformation, e.g., cysteine residues. Proteins and muteins produced by such deletions and/or insertions come within the purview of the present invention.

Preferably, the conservative amino acid groups are those defined in Table 2. More preferably, the synonymous amino acid groups are those defined in Table 3; and most preferably the synonymous amino acid groups are those defined in Table 4.

TABLE 2
Preferred Groups of Synonymous Amino Acids

| Amino Acid | Synonymous Group |
|---|---|
| Ser | Thr, Gly, Asn |
| Arg | Gln, Lys, Glu, His |
| Leu | Ile, Phe, Tyr, Met, Val |
| Pro | Gly, Ala, Thr |
| Thr | Pro, Ser, Ala, Gly, His, Gln |
| Ala | Gly, Thr, Pro |
| Val | Met, Tyr, Phe, Ile, Leu |
| Gly | Ala, Thr, Pro, Ser |
| Ile | Met, Tyr, Phe, Val, Leu |
| Phe | Trp, Met, Tyr, Ile, Val, Leu |
| Tyr | Trp, Met, Phe, Ile, Val, Leu |
| Cys | Ser, Thr |
| His | Glu, Lys, Gln, Thr, Arg |
| Gln | Glu, Lys, Asn, His, Thr, Arg |
| Asn | Gln, Asp, Ser |
| Lys | Glu, Gln, His, Arg |
| Asp | Glu, Asn |
| Glu | Asp, Lys, Asn, Gln, His, Arg |
| Met | Phe, Ile, Val, Leu |

TABLE 3
More Preferred Groups of Synonymous Amino Acids

| Amino Acid | Synonymous Group |
|---|---|
| Arg | His, Lys |
| Leu | Ile, Phe, Met |
| Pro | Ala |
| Ala | Pro |
| Val | Met, Ile |
| Ile | Met, Phe, Val, Leu |
| Phe | Met, Tyr, Ile, Leu |
| Tyr | Phe |
| Cys | Ser |
| His | Gln, Arg |
| Gln | Glu, His |
| Asn | Asp |
| Lys | Arg |
| Asp | Asn |
| Glu | Gln |
| Met | Phe, Ile, Val, Leu |

TABLE 4
Most Preferred Groups of Synonymous Amino Acids

| Amino Acid | Synonymous Group |
|---|---|
| Leu | Ile, Met |
| Ile | Met, Leu |
| Cys | Ser |
| Met | Ile, Leu |
| Trp | Met |

In a fifth aspect, the invention relates to the use of blue dye affinity chromatography and one or more steps of affinity chromatography, cation exchange chromatography, anion exchange chromatography, hydrophobic interaction chromatography, size exclusion chromatography, nanofiltration, or ultrafiltration for the separation of a single protein fused to an Fc region from an Fc-fusion proteins preparation and from free Fc-moieties.

In a sixth aspect, the invention relates to a single protein fused to an Fc region purified by the method according to the invention. In a preferred embodiment the purified single protein fused to an Fc region is a single IFN-beta fused to an Fc region (i.e. Fc arm/IFNβ-Fc arm).

In a seventh aspect, the invention relates to a single IFN-beta fused to an Fc region purified by the method according to the invention, in which the Fc region is a mutated Fc region (i.e. Fcmut/IFNβ-Fcmut).

Such purified single protein fused to an Fc region is preferably highly purified single protein fused to an Fc region. Highly purified single protein fused to an Fc region is determined e.g. by the presence of a single band in a silver-stained, non-reduced SDS-PAGE-gel after loading of protein in the amount of 1 or 2 μg per lane. Purified single protein fused to an Fc region may also be defined as eluting as a single peak in HPLC.

The preparation of purified single protein fused to an Fc region obtained from the method of the invention may contain less than 20% of impurities, preferably less than 10%, 5%, 3%, 2% or 1% of impurities, or it may be purified to homogeneity, i.e. being free from any detectable proteinaceous contaminants.

Purified single protein fused to an Fc region may be intended for therapeutic use, in particular for administration to human patients. If purified single protein fused to an Fc region is administered to patients, it is preferably administered systemically, and preferably subcutaneously, intramuscularly, epithelially e.g. via airway, or topically, i.e. locally. Rectal or intrathecal administration may also be suitable, depending on the specific medical use of purified single protein fused to an Fc region.

For administration purpose, in a preferred embodiment of the present invention, the purified single protein fused to an Fc region may be formulated into pharmaceutical composition, i.e. together with a pharmaceutically acceptable carrier, excipients or the like.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, intracranial, epidural, topical, rectal, and intranasal routes. Any other therapeutically efficacious route of administration can be used, for example absorption through epithelial or endothelial tissues such as the deep lung epithelium for airway administration. It can also be administered by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the single protein fused to an Fc region can be formulated as a solution, suspension, emulsion or lyophilised powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The “therapeutically effective amount” of the active protein(s) will be a function of many variables, including the type of single protein fused to an Fc region, the affinity of the single protein fused to an Fc region for its ligand, the route of administration, the clinical condition of the patient.

The dosage administered, as single or multiple doses, to an individual will vary depending upon a variety of factors, including pharmacokinetic properties of the single protein fused to an Fc region, the route of administration, patient conditions and characteristics (sex, age, body weight, health, size), extent of symptoms, concurrent treatments, frequency of treatment and the effect desired. Adjustment and manipulation of established dosage ranges are well within the ability of those skilled in the art, as well as in vitro and in vivo methods of determining the inhibition of the natural ligand of the therapeutic moiety in an individual.

Purified single protein fused to an Fc region may be used in an amount of about 0.001 to 100 mg/kg or about 0.01 to 10 mg/kg or body weight, or about 0.1 to 5 mg/kg of body weight or about 1 to 3 mg/kg of body weight or about 2 mg/kg of body weight.

In further preferred embodiments, the purified single protein fused to an Fc region is administered daily or every other day or three times per week or once per week.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning a range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLES

### List of Abbreviations Frequently Used Throughout the Examples

BV: bed volume
CHO: Chinese Hamster Ovary
Cond.: Conductivity
HCPs: Host Cell Proteins
INFβ-Fcmut proteins: Fcmut/IFNβ-Fcmut heterodimer and IFNβ-Fcmut/IFNβ-Fcmut homodimer described below
KCl: Potassium chloride
kDa: kilo Dalton
NaCl: Sodium chloride
OD: Optical density
PES: PolyEtherSulfone
PG: Propylene Glycol
q.s.: Quantity sufficient
RT: Room Temperature
SDS-PAGE: Sodium Dodecyl Sulphate Polyacrylamide Gel Electrophoresis
RP-HPLC: Reverse Phase-High Performance Liquid Chromatography
UV: Ultra-Violet

Equipment

PG 200/500 column (GE Healthcare)

MC126 Conductometer (Mettler-Toledo) calibrated for values at 25° C.

MP120 pH meter (Mettler Toledo)

Fcmut/INFβ-Fcmut heterodimer is an Fc-fusion fusion protein created by the fusion of an Interferon-beta protein and of an Fc domain. The Fc domain, also called Fc fragment or region, of an immunoglobulin consists of two identical arms which comprise the hinge region (H) and the second (CH2) and third (CH3) domain of an antibody heavy chain. The Fcmut/INFβ-Fcmut contains two subunits, the first comprising a mutated IgG Fc arm linked to a single IFN-β protein (SEQ ID: NO 1) and the second subunit comprising a mutated IgG Fc arm (amino acids 167 to 393 of SEQ ID: NO 1). The mutated IgG Fc arm (amino acids 167 to 393 of SEQ ID: NO 1) contains the following mutations:

| | Amino acid position in | EU index positions as |
|---|---|---|
| Mutation | SEQ ID NO: 1 | defined by Kabat et al. |
| L → A | 180 | 234 |
| L → E | 181 | 235 |
| G → A | 183 | 237 |
| A → S | 276 | 330 |
| P → S | 277 | 331 |

A CHO clone has been established for the expression of the Fcmut/INFβ-Fcmut (referred to as IFNβ-Fcmut proteins expressing CHO clone). As both subunits of Fcmut/INFβ-Fcmut are expressed by the clone, various species/forms of the proteins are produced and are designated as follows:
“Free Fc” (Fcmut/Fcmut dimer)
“Fcmut/INFβ-Fcmut” (Fcmut/INFβ-Fcmut heterodimer)
“IFN-beta dimer” (INFβ-Fcmut/INFβ-Fcmut homodimer)
Aggregates and truncated forms.

RP-HPLC analysis on the cell culture supernatant produced in simple batch mode showed that the “Fcmut/INFβ-Fcmut” accounts for approximately 50% of the total molecules produced. The “free Fc” represents approximately 30% and the “IFN-beta dimer” represents approximately 20% of the total molecules produced.

Purification processes were established for the purification of Fcmut/INFβ-Fcmut from the other forms produced. The Fcmut/INFβ-Fcmut has a molecular weight of approximately 70 kDa and a pl around 7-7.5. This purification processes include a blue sepharose chromatography step allowing the reduction of free Fc to very low levels.

### Example 1

### Step 1: Capture Step on Protein a and Filtration of the Post Capture Eluate

Clarified harvest of an IFNβ-Fcmut proteins expressing CHO clone cultured under serum-free conditions was first subjected to Protein A affinity chromatography as capture step, in particular for the elimination of HCPs and INFβ-Fcmut proteins aggregates. The post capture eluate was defrosted at room temperature and filtrated on Millistak+A1 HC filter (reference MA1HC01FS1; Millipore). The filtration cassette was pre-washed with the equilibration buffer 1 described in step 2 below (at least 100 liter/m²). After the filtration, the filter and filtration system were washed with 1.5 to 2 times the dead volume. This wash solution was collected with the filtered post capture eluate and the mix was subjected to the blue sepharose chromatography.

### Step 2: Blue Sepharose Chromatography

The blue sepharose chromatography was developed for the removal of free Fc fraction produced.

All the operations were performed at room temperature (20±5° C.) and the flow rate was kept constant at 300 cm/h except during the elution were it was at 160 cm/h due to the high viscosity of the elution buffer. The UV signal at 280 nm was recorded at all time.

### Equipment: a) Buffers

| Buffer 1 | Sodium acetate 3H₂O | 4.34 | g/kg |
|---|---|---|---|
| Equilibration & | Acetic acid | 1.1 | ml/kg |
| Wash 1 buffer | Specifications | pH: 5.0 ± 0.2 | |
| (50 mM Sodium | (pH)/Mean (Cond.) | Conductivity: | |
| Acetate pH 5.0) | prior to filtration | 2.5 ± 0.2 mS/cm | |
| Buffer 2 | Ammonium acetate | 3.85 | g/kg |
| Wash 2 buffer | Adjustment with | q.s. pH 8.7 ± 0.1 | |
| (50 mM ammonium | ammonia at 25% | | |
| acetate pH 8.7) | Specifications | pH: 8.7 ± 0.1 | |
| | (pH)/Mean (Cond.) | Conductivity: | |
| | prior to filtration | 4.9 ± 0.5 mS/cm | |
| Buffer 3 | Ammonium acetate | 3.85 | g/kg |
| Elution buffer | NaCl | 18.46 | g/kg |
| (Ammonium acetate, | Propylene glycol | 485 | g/kg |
| NaCl, Propylene | Adjustment with | q.s. pH 8.5 ± 0.2 | |
| glycol, pH 8.5) | ammonia at 25% | | |
| | Specifications | Conductivity: | |
| | (pH)/Mean (Cond.) | 8.0 ± 0.5 | |
| | prior to filtration | mS/cmpH: 8.5 ± 0.2 | |
| Buffer 4 | NaOH | 19.6 | g/kg |
| Regeneration/ | | | |
| sanitisation buffer | | | |

| | | |
|---|---|---|
| Buffer 5 | Ethanol 20% | 20 ml/100 ml |
| Storage Buffer | | |

b) Column

A BPG 200/500 column (GE Healthcare) was packed with Blue Sepharose 6FF resin (GE Healthcare) to a bed height of 8±1 cm (about 2.5 litres of resin).

The following passages were followed to perform the blue sepharose chromatography (see also Table 5):

Equilibration

The column was equilibrated with at least 6 BV of buffer 1 until the conductivity and pH of the flow through reached the values of buffer 1 (i.e. pH: 5.0±0.2 and a conductivity of 2.5±0.2 mS/cm).

Loading

The column was loaded with the filtered post-capture eluate at pH 4.5 at a maximum dynamic capacity of about 40 g of IFNβ-Fcmut proteins (RP-HPLC analysis) per litre of packed resin. Various runs were performed using different load capacities (see table 6 below).

Wash 1

The column was washed with at least 5 BV of buffer 1 until a stable baseline was reached.

Wash 2

The column was washed with at least 5 BV of buffer 2 until the appearance of a peak of absorbance at 280 nm, corresponding to free Fc. Washing was continued until a stable baseline was reached.

Elution

The flow rate was lowered to 160 cm/h.

Elution was achieved with at least 9 BV of buffer 3 and the eluate collected in a sterile container. When the BV of buffer is applied to the column, a small absorbance peak can appear at to 280 nm. This small peak should preferably not be collected at it is a residual quantity of free Fc. The eluate, corresponding to IFNβ-Fcmut proteins, is then collected when a major rise of the peak of absorbance appears until a stable baseline is reached.

Wash 3

The column was washed with at least 5 BV of water. The flow rate initially at 160 cm/h was increased up to 300 cm/h.

Regeneration & Sanitisation

The column was sanitised with at least 3 BV of buffer 4 followed by a stand-by of 30 to 60 minutes. The resin was then washed with about 10 BV of purified water until a return of the pH to 7.0.

TABLE 5
Summary table reporting chromatography passages and buffers

| | | | | Second | | |
|---|---|---|---|---|---|---|
| Step | Equilibration | Load | First washing | washing | Elution | Regeneration |
| Buffer | 50 mM | NA | 50 mM | 50 mM | 50 mM | NaOH 0.5 M |
| | Sodium | | Sodium | ammonium | ammonium | |
| | Acetate | | Acetate | acetate | acetate | |
| pH | 5 | 4.5 | 5 | 8.7 | 8.5 | >12 |
| conductivity | 2.5 | NA | 2.5 | 4.9 | 8 | NA |
| mS/cm | | | | | | |
| [salt | 0 | 0 | 0 | 0 | 18.46 | 0 |
| concentration] | | | | | | |
| g/kg | | | | | | |
| [propylene | 0 | 0 | 0 | 0 | 48.5 | 0 |
| glycol] % | | | | | | |
| BV (at least) | 6 | NA | 5 | 5 | 9-11 | 3 |
| linear velocity | 300 | 300 | 300 | 300 | 160 | 300 |
| (cm/hr) | | | | | | |

Storage

The column was washed with buffer 5 and stored at room temperature.

Various runs were performed using different load capacities.

### Results

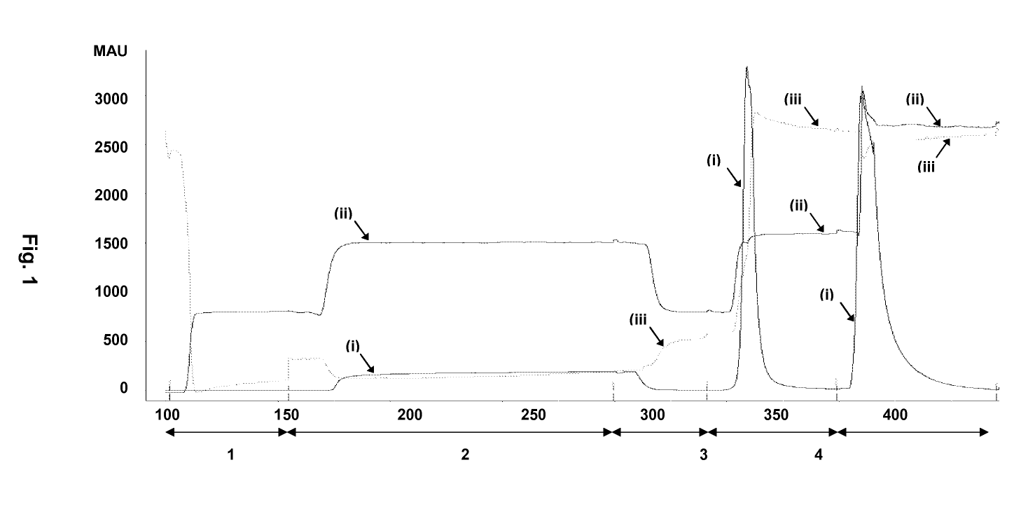

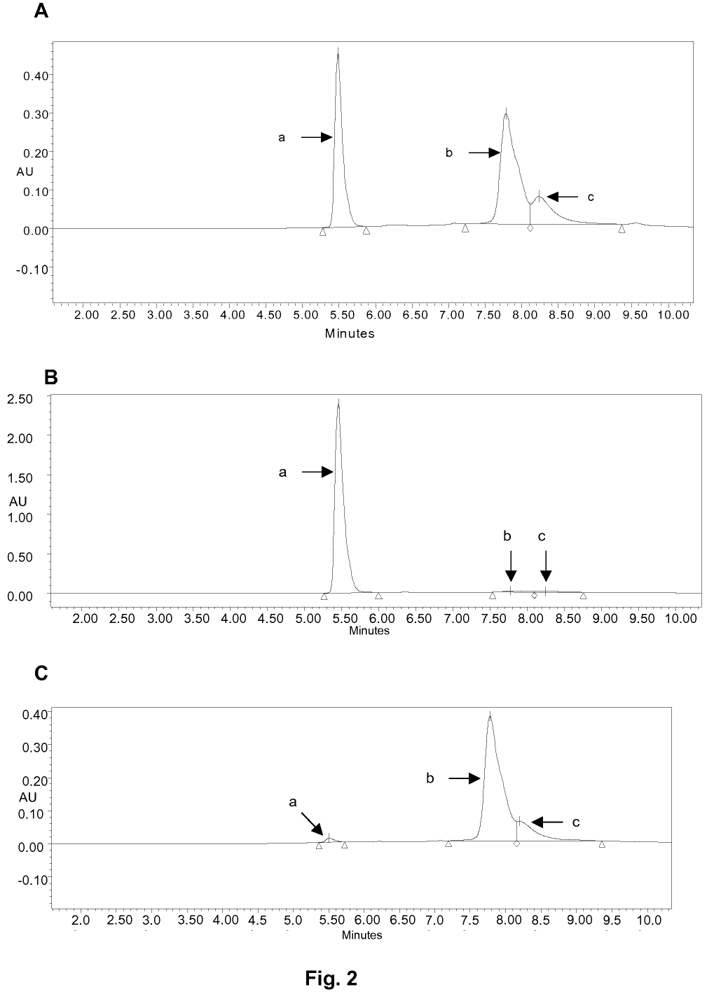

FIG. 1 shows the chromatographic profile of Run 67 (load capacity tested 18 g/L, Table 6). Two distinct peaks are resolved using the conditions described for passages “wash 2” and “elution”. The peak of “wash 2” was identified as the free Fc fraction. The peak of “elution” represents the purified IFNβ-Fcmut proteins (i.e. Fcmut/IFNβ-Fcmut and IFN-beta dimer). The conditions used in “wash 2”, i.e. 50 mM ammonium acetate at pH: 8.7±0.1, resulted in the reduction of the free Fc fraction to 1-2%. As shown in FIG. 2.C, the eluate from the blue sepharose, containing the Fcmut/IFNβ-Fcmut and the IFN-beta dimer, was found to be free of Fc moieties. The Blue Sepharose chromatography also permits an additional reduction of the HCPs content. Table 6 below reports the content of free Fc in different fractions for various runs.

TABLE 6
Free Fc content in the load, Wash 2 and eluate fractions for various runs

| Fractions | Run 54 | Run 60 | Run 64 | Run 67 | Run 78 | Run 79 |
|---|---|---|---|---|---|---|
| Load | 37% | 39% | 35% | 35% | 36% | 36% |
| Washing 2 | 98% | 99% | 98% | 99% | 98% | 98% |
| Eluate | 2% | 1% | 2% | 1% | 2% | 2% |
| Capacity tested | 12 | 17 | 16 | 18 | 16 | 16 |
| g/L | | | | | | |

### Step 3: Dialysis of the blue sepharose eluate

The eluate of the blue sepharose column of Step 2 above was dialysed into a suitable buffer (Sodium acetate, NaCl, pH 5.0) in order to reduce the concentration of propylene glycol. The following buffers and cassette were used:
a) cassette
Kvick Flow Cassette (GE Healthcare, reference 56-4113-51) with 0.46 m² membrane surface and a porosity of 30 Kda in PES (polyethersulfone). 4 was the number of cassettes necessary for the dialysis;
b) buffers

| Dialysis Buffer | Sodium acetate 3H₂O | 4.34 | g/kg |
|---|---|---|---|
| (Sodium | water-free acetic acid | 1.1 | ml/kg |
| acetate, NaCl, | (glacial acetic acid) | | |
| pH 5.0) | NaCl | 2.86 | g/kg |
| | Specifications | pH: 5.0 ± 0.2 | |
| | (pH)/Mean (Cond.) | Conductivity: | |
| | prior to filtration | 7.5 ± 0.2 mS/cm | |
| | | (conductivity at 25° C.) | |
| Cleaning buffer | NaOH | 19.6 | g/kg |
| | 48° solution of sodium | 2.5 | ml/kg |
| | hypochlorite (bleach) | | |
| Storage buffer | NaOH | 4 | g/kg |

### Results

The mean yield in OD and RP-HPLC of this step was between 80 and 100%.

### Step 4: Cation Exchange Chromatography on Fractogel SO₃—

All the operations were performed at room temperature (20±5° C.) and the flow rate was kept constant at 240 cm/h except at the beginning of the equilibration, at the final wash (purified water) and storage phases where it was at 125 cm/h due to the pressure increase. The UV signal at 280 nm was recorded at all time.

Equipment: a) buffers

| Equilibration and | Sodium acetate 3H₂O | 4.34 | g/kg |
|---|---|---|---|
| Wash buffer | water-free acetic acid | 1.1 | ml/kg |
| (Sodium acetate, | (glacial acetic acid) | | |
| NaCl, pH 5.0) | NaCl | 2.86 | g/kg |
| | Specifications | pH: 5.0 ± 0.2 | |
| | (pH)/Mean (Cond.) | Conductivity: | |
| | prior to filtration | 7.5 ± 0.2 mS/cm | |
| | | (conductivity at 25° C.) | |
| Elution buffer | NaH₂PO₄, 2H₂O | 2.34 | g/kg |
| (d = 1.003) | Na₂HPO₄, 2H₂O | 4.45 | g/kg |
| (40 mM sodium | KCl | 7.46 | g/kg |
| phosphate, 100 mM | Specifications | pH: 7.0 ± 0.1 | |
| KCl, pH 7.0) | (pH)/Mean (Cond.) | Conductivity: | |
| | prior to filtration | 16 ± 0.5 mS/cm | |
| | | (Conductivity at 25° C.) | |
| Regeneration buffer | NaCl | 83.01 | g/kg |
| Sanitisation buffer | NaOH | 19.6 | g/kg |

| | | |
|---|---|---|
| Storage buffer | Ethanol 20% | 20 ml/100 ml |

b) column
A BPG 200 column (GE Healthcare) was packed with Fractogel S03 (Merck, reference 1.14894) to a bed height of 8±1 cm (about 2.5 litres of resin).

The Cation exchange chromatography was performed as follows.

Equilibration

The column was equilibrated with at least 9 BV of equilibration buffer until the conductivity and pH of the flow through reached the values of the buffer. The flow rate was gradually increased from 125 cm/h to 240 cm/h according to the evolution of the pressure.

Loading

The column was loaded with the post blue sepharose eluate dialysed as described in Step 3 at a maximum dynamic capacity of 20 g of IFNβ-Fcmut proteins (OD 280 nm) per litre of packed resin.

Wash

The column was washed with at least 4 BV of the equilibration/wash buffer 1 until a stable baseline was reached.

Elution

Elution was achieved with at least 10 to 11 BV of the elution buffer and the eluate collected in a sterile container. When the BV of buffer is applied to the column, a small absorbance peak can appear at 280 nm. This small peak should preferably not be collected. The eluate, containing the Fcmut/IFNβ-Fcmut fraction, is then collected when a major rise of the peak of absorbance appears until a stable baseline of the UV signal is reached.

Regeneration

This stage corresponds to the elimination of the IFN-beta dimers and aggregates. The column was regenerated with at least 8 BV of the regeneration buffer, until a stable baseline of the UV signal is reached.

Sanitisation

The column was sanitized with at least 3 BV of sanitization buffer followed by a stand-by of 30 to 60 minutes. The flow rate was gradually lowered to 125 cm/h. The resin was washed with purified water until the pH reached 7 (control with pH paper).

Storage

The column was washed with at least 2 BV the storage buffer and stored at room temperature.

### Results

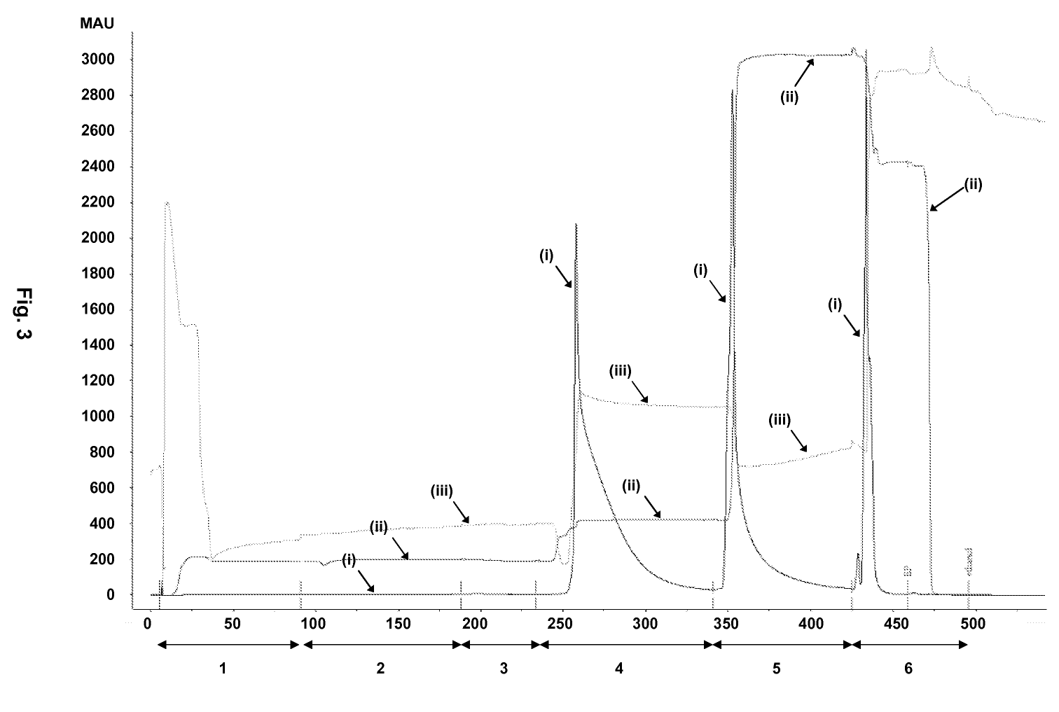

FIG. 3 shows the chromatographic profile of the cation exchange chromatography. The elution buffer used has the capacity to separate the Fcmut/IFNβ-Fcmut on one hand and the IFN-beta dimers and aggregates on the other hand. Thus the elution peak represents the Fcmut/IFNβ-Fcmut fraction whereas the regeneration peak represents the IFN-beta dimers and aggregates. When the conductivity was increased (up to 25 mS/cm), the separation of the Fcmut/IFNβ-Fcmut and IFN-beta dimers was less effective (results not shown). The Cation Exchange chromatography also permits an additional reduction of the HCPs content.

### Step 5: Hydrophobic Interaction Chromatography on PPG-600M

All the operations were performed at room temperature (20±5° C.) and the flow rate was kept constant at 130 cm/h. The UV signal at 280 nm was recorded at all time.

Equipment: a) column
A BPG 200/500 column (GE Healthcare) was packed with PPG-600M (Tosoh, reference 21304) to a bed height of 11±1 cm (about 3.5 litres of resin).
The column is used in opposite mode (not-retained) where the fractions of interest are not bound to the column but are collected in the flow through.

b) buffers

| Equilibration/ | NaH₂PO₄, 2H₂O | 1.488 | g/kg |
|---|---|---|---|
| Wash buffer | Na₂HPO₄, 2H₂O | 5.094 | g/kg |
| (40 mM sodium | Ammonium Sulphate | 63.04 | g/kg |
| phosphate, | Mean (pH)/Mean | pH: 6.8 ± 0.2 | |
| 0.5M ammonium | (Cond.) prior | Conductivity: | |
| sulphate, pH 6.8) | to filtration | 75.3 ± 2 mS/cm | |
| | | (Conductivity at 25° C.) | |
| Sanitisation | NaOH | 19.6 | g/kg |
| buffer | | | |

| | | |
|---|---|---|
| Storage buffer | Ethanol 20% | 20 ml/100 ml |

The following passages were followed to perform the Hydrophobic Interaction chromatography:

Equilibration

The column was equilibrated with at least 5 BV of the equilibration/wash buffer until the conductivity and pH of the flow through reached the values of the buffer.

Loading

The column was loaded with the eluate of the Fractogel column obtained in Step 4 described above to which 66.07 g/kg of ammonium sulphate was added. The preparation obtained was loaded on the resin at a maximum capacity of 10 g of IFNβ-Fcmut proteins, now containing almost only Fcmut/IFNβ-Fcmut, (OD280 nm) per liter of packed resin.

Wash

The column was washed with at least 7 BV of the equilibration/wash buffer until a stable baseline of the UV signal was reached. The flow through, containing the purified Fcmut/IFNβ-Fcmut, was collected when a major rise of the peak of absorbance at 280 nm appeared and until a stable baseline was reached at the end of the wash.

Regeneration

The column was regenerated with at least 3 BV of the regeneration buffer, until a stable baseline of the absorption peak at 280 nm corresponding to the aggregates and HCPs was reached.

Sanitisation

The column was sanitized with at least 3 BV of sanitization buffer followed by a stand-by of 30 to 60 minutes. The resin was washed with purified water until the pH reached 7 (control with pH paper).

Storage

The column was washed with at least 3 BV the storage buffer and stored at room temperature.

### Results

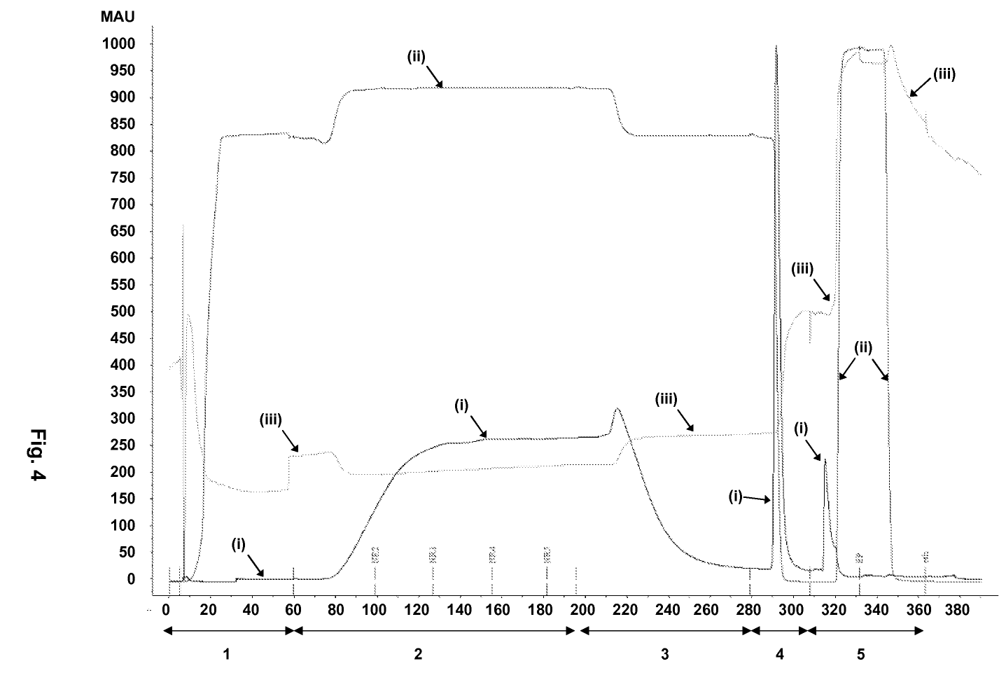

FIG. 4 shows the chromatographic profile of hydrophobic interaction chromatography wherein the protein of interest (i.e. Fcmut/IFNβ-Fcmut) was present in the unbound fraction at the load step and in the flow though of the wash step. The maximum load capacity tested was of 24 g (OD280 nm)/L, however the resin selectivity for the aggregates and HCPs was better when the load capacity used was lower (i.e. 5-10 g/L). The mean yield of this step in OD was at about 90%. The hydrophobic interaction chromatography permits the elimination of the remaining HCPs and aggregates.

### Step 6: Nanofiltration

The fraction collected at step 5 was subjected to nanofiltration on a ViroSart CPV filter (Sartorius).

### Step 7: Concentration/Final Dialysis

The flow through of the hydrophobic interaction chromatography column containing the purified Fcmut/IFNβ-Fcmut was dialysed against a citrate buffer at pH 6.0 and concentrated to 3 g/l. This buffer minimizes the generation of aggregates.

### CONCLUSION

Efficient purification processes, including a step allowing the reduction of free Fc to very low levels, were established for the purification of Fcmut/INFβ-Fcmut. The specific wash conditions developed for the blue sepharose chromatography efficiently reduced the amount of the free Fc fraction. The chromatography steps applied i.e. cation exchange chromatography followed by hydrophobic interaction chromatography allowed obtaining the protein of interest (i.e. Fcmut/INFβ-Fcmut) to homogeneity.

### REFERENCES

1. Altschul, S. F., Gish, W., Miller, W., Myers, E. W., and Lipman, D. J. (1990). Basic local alignment search tool. J. Mol. Biol. 215, 403-410.
2. Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
3. Armour K L, Clark M R, Hadley A G, Williamson L M. (1999). Recombinant human IgG molecules lacking Fc-gamma receptor I binding and monocyte triggering activities. Eur J. Immunol. 29(8): 2613-24
4. Ausubel et al., Current Protocols in Molecular Biology, supra, Interscience, N.Y., §§6.3 and 6.4 (1987, 1992).
5. Bodmer J L, Schneider P, Tschopp J. (2002). The molecular architecture of the TNF superfamily. Trends Biochem Sci. 2002 January; 27(1):19-26.
6. Bossen C, Ingold K, Tardivel A, Bodmer J L, Gaide O, Hertig S, Ambrose C, Tschopp J, Schneider P. (2006). Interactions of tumor necrosis factor (TNF) and TNF receptor family members in the mouse and human. J Biol Chem. 2006 May 19; 281(20):13964-71.
7. Carter P J. (2006). Potent antibody therapeutics by design. Nat Rev Immunol. 2006 May; 6(5):343-57.
8. Devereux, J., Haeberli, P., and Smithies, O. (1984). A comprehensive set of sequence analysis programs for the VAX. Nucleic Acids Res. 12, 387-395.
9. Grantham, R. (1974). Amino acid difference formula to help explain protein evolution. Science 185, 862-864.
10. Hinton P R, Johlfs M G, Xiong J M, Hanestad K, Ong K C, Bullock C, Keller S, Tang M T, Tso J Y, Vasquez M, Tsurushita N. (2004). Engineered human IgG antibodies with longer serum half-lives in primates. J Biol Chem. 279(8): 6213-6.
11. Idusogie E E. et al. (2000). Mapping of the C1q binding site on rituxan, a chimeric antibody with a human IgG1 Fc. J Immunol. 164(8):4178-84.
12. Idusogie E E. et al. (2001). Engineered antibodies with increased activity to recruit complement. J Immunol. 166(4): 2571-5.
13. Kabat, E. A., Wu, T. T., Perry, H. M., Gottesman, K. S., and Foeller, C. (1991), Sequences of Proteins of Immunological Interest, 5th Ed., National Institutes of Health, Bethesda, Md.
14. Knight E Jr and Fahey (1981). Human fibroblast interferon. An improved purification. J Biol Chem. 256(8): 3609-11.
15. Locksley et al. (2001). The TNF and TNF receptor superfamilies: integrating mammalian biology. Cell 104, 487-501.
16. Moore et al., (1999). BLyS: member of the tumor necrosis factor family and B lymphocyte stimulator. Science. 285(5425): 260-3.
17. Naismith J H, Sprang S R. (1998). Modularity in the TNF-receptor family. Trends Biochem Sci. 23(2):74-9. Review.
18. Novak et al. (2004). Expression of BCMA, TACI, and BAFF-R in multiple myeloma: a mechanism for growth and survival. Blood. 103(2): 689-94. Epub 2003 Sep. 25.
19. Shields R L. et al. (2001). High resolution mapping of the binding site on human IgG1 for Fc gamma RI, Fc gamma RII, Fc gamma RIII, and FcRn and design of IgG1 variants with improved binding to the Fc gamma R. J Biol Chem. 276(9): 6591-604.
20. Smith, T. F. and Waterman, M. S. (1981). Identification of common molecular subsequences. J. Mol. Biol. 147, 195-197.
21. Steurer W, Nickerson P W, Steele A W, Steiger J, Zheng X X, Strom T B. (1995). Ex vivo coating of islet cell allografts with murine CTLA4/Fc promotes graft tolerance. J Immunol. 155(3): 1165-74
22. Vaccaro C, Zhou J, Ober R J, Ward E S. (2005). Engineering the Fc region of immunoglobulin G to modulate in vivo antibody levels. Nat Biotechnol. 23(10): 1283-8.
23. Vlatakis G, Skarpelis G, Stratidaki I, Bouriotis V, Clonis Y D. (1987). Dye-ligand chromatography for the resolution and purification of restriction endonucleases. Appl Biochem Biotechnol. 15(3):201-12.
24. WO 2005/001025
25. EP1230354

1-33. (canceled)

34. A method for purifying an Fc-fusion proteins preparation containing a single protein fused to an Fc region from free Fc-moieties present in a fluid comprising said Fc-fusion proteins, the method comprising the steps of:
(a) loading said fluid on a blue dye affinity chromatography resin;
(b) washing the resin with a buffer having a pH of about 8.4 to about 8.9 to wash the free Fc-moieties from the resin; and
(c) eluting the Fc-fusion proteins from the resin.

35. The method according to claim 34, wherein the Fc-fusion proteins are able to bind to a blue dye affinity chromatography resin.

36. The method according to claim 34, wherein the blue dye affinity chromatography of step (a) is carried out with a resin having immobilised Cibacron Blue F3G-A.

37. The method according to claim 36, wherein the resin comprises Cibacron Blue F3G-A immobilized on agarose.

38. The method according to claim 34, wherein step (a) comprises loading the blue sepharose resin at a maximum dynamic capacity of 40 g of Fc-fusion proteins per liter of packed blue sepharose resin.

39. The method according to claim 38, wherein the fluid in step (a) is loaded on the resin at about pH 5.

40. The method according to claim 34, wherein step (b) is carried out using an ammonium acetate buffer at a pH of 8.7±0.2.

41. The method according to claim 40, wherein the ammonium acetate is at a concentration of 50 mM.

42. The method according to claim 34, wherein step (c) is carried out using an elution buffer at pH 8.5±0.2 comprising ammonium acetate and propylene glycol.

43. The method according to claim 34, wherein the eluate of the blue dye affinity chromatography resin resulting from step (c) has levels of free Fc-moieties that are undetectable by SDS-PAGE under non-reducing conditions and silver staining when loading 1 μg of Fc-fusion proteins.

44. The method according to claim 34, wherein in step (a) the fluid comprising said Fc-fusion proteins is a filtrated Protein A chromatography eluate.

45. The method according to claim 34, wherein the Fc-fusion proteins comprise part of an Immunoglobulin (Ig) constant region.

46. The method according to claim 45, wherein the constant region is a human constant region.

47. The method according to claim 45, wherein the immunoglobulin is an IgG1.

48. The method according to claim 45, wherein the constant region comprises at least a CH2 and a CH3 domain.

49. The method according to claim 45, wherein the Fc-fusion proteins comprise a single protein fused to an Fc region.

50. The method according to claim 49, wherein the Fc-fusion proteins comprise a single protein fused to an Fc region and said single protein comprises a polypeptide selected from the group consisting of:
(a) SEQ ID NO: 1 or 3;
(b) a polypeptide encoded by a polynucleotide hybridising to the complement of SEQ ID NO: 2 or 4 under highly stringent conditions; and
(c) a mutein of (a) having at least 80% or 85% or 90% or 95% sequence identity to the polypeptide of (a).

51. The method according to claim 34, further comprising one or more additional steps of affinity chromatography, cation exchange chromatography, anion exchange chromatography, hydrophobic interaction chromatography, size exclusion chromatography, nanofiltration, or ultrafiltration.

52. The method according to claim 51, wherein one or more of said additional steps permit the separation of a single protein fused to an Fc region from an Fc-fusion proteins preparation.

53. The method according to claim 52, wherein the single protein fused to an Fc region is able to bind to a blue dye affinity chromatography resin.

54. The method according to claim 52, wherein the single protein fused to an Fc region is a single IFN-β fused to an Fc region.

55. A method for purifying a polypeptide comprising:
(a) subjecting a fluid comprising the polypeptide to Protein A chromatography;
(b) subjecting the eluate of step (a) to blue dye chromatography;
(c) subjecting the eluate of step (b) to cation exchange chromatography;
(d) subjecting the eluate of step (c) to hydrophobic interaction chromatography; and
(e) collecting the flow through of the hydrophobic interaction chromatography of step (d).

56. A method for purifying a polypeptide comprising:
(a) subjecting a fluid comprising the polypeptide to Protein A chromatography;
(b) subjecting the eluate of step (a) to blue dye chromatography;
(c) subjecting the eluate of step (b) to anion exchange chromatography;
(d) subjecting the eluate of step (c) to size exclusion chromatography; and
(e) collecting the eluate of the size exclusion chromatography step (d).

57. The method according to claim 51, further comprising formulating the single protein fused to an Fc region into a pharmaceutical composition.

58. Fc-fusion proteins purified by the method according to claim 34.

59. The purified Fc-fusion proteins according to claim 58 comprising less than about 5% (new) free Fc-moieties of the total protein concentration.

60. The purified Fc-fusion proteins according to claim 58, which consist of a single IFN-β fused to an Fc region.

61. The purified Fc-fusion proteins of claim 60, wherein the Fc region is a mutated Fc region.

62. A pharmaceutical composition comprising the Fc-fusion proteins according to claim 60.
